(19) United States
(12) Patent Application Publication
(10) Pub. No.: US 2004/0052737 A1
(43) Pub. Date: Mar. 18, 2004

(54) Emollient particles, sunscreen compositions and methods of manufacture thereof

(76) Inventor: John C. Hill, Mesa, AZ (US)

Correspondence Address: PARSONS & GOLTRY, Suite 260, 340 East Palm Lane, Phoenix, AZ, 85004, US

(21) Appl. No.: 10/127,933

(22) Filed: Apr. 23, 2002

(51) Int. Cl. A61K 7/42

(52) U.S. Cl. 424/59

(57) ABSTRACT

Compositions consisting of emollient particles that carry at least one of active sunscreen material, self-tanning material and visual effects material, and transparent and translucent topical sunscreen preparations having disposed therein such emollient particles.

### FIELD OF THE INVENTION

This invention relates to topical preparations and, more particularly, to sunscreen compositions and associated application methods and methods of manufacture.

### BACKGROUND OF THE INVENTION

Topical sunscreen preparations capable of protecting the skin from the damaging effects of excessive ultraviolet (UV) radiation use active ingredients to block, absorb and/or reflect this radiation. Skilled artisans provide high sun protection factor (SPF) lotions, gels, and creams to provide the skin with the highest available protection against burning as a result of prolonged exposure to UV radiation. In order to provide the skin with the highest available protection against UV radiation, it is necessary to use active ingredients that are capable of blocking UV radiation, namely, white, solid, inorganic material such as titanium dioxide, zinc oxide, and the like. Such UV radiation blocking materials are insoluble in the carrier material of existing sunscreen formulations and are simply suspended therein, leaving the final topical sunscreen preparation with a white, chalky, opaque appearance. Furthermore, because such inorganic UV radiation blocking material must be suspended, only a limited amount can be used in a topical sunscreen preparation, limiting the UV radiation-blocking characteristic of the resulting topical sunscreen preparation.

Given these and other deficiencies in the art, the need for certain new and useful improvements is evident.

### SUMMARY OF THE INVENTION

The above problems and others are at least partially solved and the above purposes and others realized in improved sunscreen compositions and associated application and manufacturing. A preferred composition embodiment of the invention consists of emollient particles and active sunscreen material carried by the emollient particles. Further to this embodiment is a topical sunscreen preparation, which is one of transparent and translucent, into which are disposed the emollient particles. The active sunscreen material consists of titanium dioxide, zinc oxide or other like material or combination of materials. Still further to this embodiment is self-tanning material carried by the emollient particles, such as dihydroxyacetone or other like self-tanning material or materials. Yet still further to this embodiment is visual effects material carried by the emollient particles, such as pigment material for imparting color to the skin, glitter material for applying glittery effects to the skin, etc. Because self-tanning material imparts a tanning effect to skin, it can be considered within the ambit of visual effects material.

Another preferred composition embodiment of the invention consists of a topical sunscreen preparation, which is one of transparent and translucent, emollient particles disposed in the topical sunscreen preparation, and active sunscreen material carried by the emollient particles. The active sunscreen material consists of titanium dioxide, zinc oxide or other like material or combination of materials. Still further to this embodiment is self-tanning material carried by the emollient particles, such as dihydroxyacetone or other like self-tanning material or materials. Yet still further to this embodiment is visual effects material carried by the emollient particles, such as pigment material for imparting color to the skin, glitter material for applying glittery effects to the skin, etc. Because self-tanning material imparts a tanning effect to skin, it can be considered within the ambit of visual effects material.

Yet still a further composition embodiment of the invention consists of a topical sunscreen preparation, which is one of transparent and translucent, and emollient particles disposed in the topical sunscreen preparation and carrying at least one of a)active sunscreen material, b)self-tanning material, and c)visual effects material. The active sunscreen material consists of titanium dioxide, zinc oxide or other like material or combination of materials. The self-tanning material is dihydroxyacetone or other like self-tanning material or materials. The visual effects material is pigment material for imparting color to the skin, glitter material for applying glittery effects to the skin, etc. Because self-tanning material imparts a tanning effect to skin, it can be considered within the ambit of visual effects material.

A preferred method embodiment of the invention includes providing a topical sunscreen preparation, which is one of transparent and translucent, providing emollient particles carrying active sunscreen material, and disposing the emollient particles in the topical sunscreen preparation. The active sunscreen material consists of titanium dioxide, zinc oxide or other like material or combination of materials. Further to this embodiment, the emollient particles also carry self-tanning material, such as dihydroxyacetone or other like self-tanning material or materials. Still further to this embodiment, the emollient particles further carry visual effects material, such as pigment material for imparting color to the skin, glitter material for applying glittery effects to the skin, etc. Because self-tanning material imparts a tanning effect to skin, it can be considered within the ambit of visual effects material.

Another preferred method embodiment of the invention includes providing a topical sunscreen preparation, which is one of transparent and translucent, providing emollient particles carrying at least one of a)active sunscreen material, b)self-tanning material, and c)visual effects material, and disposing the emollient particles in the topical sunscreen preparation. The active sunscreen material consists of titanium dioxide, zinc oxide or other like material or combination of materials. The self-tanning material is dihydroxyacetone or other like self-tanning material or materials. The visual effects material is pigment material for imparting color to the skin, glitter material for applying glittery effects to the skin, etc. Because self-tanning material imparts a tanning effect to skin, it can be considered within the ambit of visual effects material.

Consistent with these and ensuing teachings, the invention also contemplates other associated composition and method embodiments.

### DETAILED DESCRIPTION OF PREFERRED EMBODIMENTS

An emollient is a substance that softens the skin by slowing water evaporation. Some emollients, such as jojoba esters, hydrogenated triglycerides, partially hydrogenated trigylcerides, petrolatum, polyethylene, ethylhexyl palmitate, and various combinations thereof, among other emollients and emollient combinations and formulations, are capable of being formed into solid emollient particles for use either alone or in combination with various types of cosmetic preparations, whether soap preparations, lotions, creams, etc. The provision of emollient particles and associated construction methods are disclosed in U.S. Pat. No. 6,280,746 B1 entitled DRY EMOLLIENT COMPOSITIONS, which is incorporated by reference herein. Emollient particles can be produced with various materials or combinations thereof including xanthum gums, gellan gums, and starches, and in various ways including agglomeration, encapsulation, chemical reactance, spray chilling, prilling, mechanical compaction, pressing, etc. In accordance with the principle of the invention, this disclosure concerns novel compositions that exploit emollient particles as carriers and deliverers of specific materials, namely, emollient particles that carry active sunscreen material, emollient particles that carry self-tanning material, emollient particles that carry visual effects material such as glitter material and coloring material, and transparent and translucent topical sunscreen preparations having disposed therein such emollient particles. Because self-tanning material imparts a tanning effect to skin, it can be considered within the ambit of visual effects material.

Instructive of exemplary and well-known topical sunscreen preparations are transparent and translucent aqueous and oil-based preparations, incorporating not only one or more active ingredients capable of blocking, absorbing and/or reflecting UV radiation, but also other various excipients, such as one or more oils, esters, surfactants, fragrance material, etc. The invention is concerned with transparent and translucent topical sunscreen preparations incorporating emollient particles that carry at least one of a) active sunscreen material, b) visual effects material, and c) self-tanning material, and, in various other particular embodiments, any combination thereof and in desired percentages of two or more by weight of the emollient formulation from which such emollient particles are formed.

In accordance with the principle of the invention in connection with a particular embodiment is the provision of emollient particles and active sunscreen material carried by the emollient particles. Preferably, the active sunscreen material is UV radiation blocking material, such as titanium dioxide, zinc oxide, a combination of titanium dioxide and zinc oxide, etc. Other like UV radiation blocking material or combination of UV radiation blocking materials can be used without departing from the invention. The emollient particles preferably incorporate approximately ten percent (10%) by weight of the active sunscreen material, and less or more can be used depending on specific needs. Emollient particles incorporating active sunscreen material are capable of being rubbed onto or otherwise applied to the skin, in which they will disappear or otherwise dissipate onto the skin leaving disposed thereon a persistent emollient layer and the active sunscreen material, protecting the skin from the harmful effects of prolonged exposure to UV radiation.

In an exemplary embodiment, emollient particles incorporating active sunscreen material in accordance with the invention are disposed in a topical sunscreen preparation that is one of transparent and translucent, such as any variety of well-known aqueous or oil-based topical sunscreen preparations or the like. Already having a pre-determined SPF factor as provided by the sunscreen material furnished with the topical sunscreen preparation, disposing emollient particles incorporating active sunscreen material as herein provided into such a topical sunscreen preparation increases the SPF factor of the topical sunscreen preparation without causing the undesirable effect of eliminating the clarity, transparency or translucency of the topical sunscreen preparation. Rather, the emollient spheres appear as particulate matter suspended in the topical sunscreen preparation, or can be made to suspend with minor adjustments of the topical sunscreen preparation formulation by one skilled in the art. Because most transparent and translucent topical sunscreen preparations incorporate suspension agents, emollient particles incorporating active sunscreen material in accordance with the invention will suspend in such topical sunscreen preparations. Additional suspension agents can be added, if desired.

Preferably, a sunscreen composition consisting of a mixture of a topical sunscreen preparation, which is one of transparent and translucent, and emollient particles carrying active sunscreen material as provided herein incorporate ninety percent (90%) by weight of the topical sunscreen preparation and ten percent (10%) by weight of the emollient particles that incorporate active sunscreen material. Other percentages can be used depending on specific needs and depending on the desired SPF factor. A topical sunscreen preparation, which is one of transparent and translucent, having disposed therein emollient particles incorporating active sunscreen material is capable of being rubbed onto or otherwise applied to the skin, in which the topical sunscreen preparation will absorb into the skin and the emollient particles will dissipate onto the skin, leaving disposed thereon the sunscreen material of the topical sunscreen preparation, a persistent emollient layer and the active sunscreen material of the emollient particles, protecting the skin from the harmful effects of prolonged exposure to UV radiation.

In accordance with another principle of the invention is the provision of emollient particles and visual effects material carried by the emollient particles capable of imparting a visual characteristic to skin. In one embodiment, the visual characteristic to skin is a tanning effect. In another embodiment, the visual characteristic is a coloring effect. In yet another embodiment, the visual characteristic is a glittery effect. One or more of the foregoing visual characteristics can be incorporated into the emollient particles of the invention, with or without active sunscreen material, and at desired percentages by weight of the formulation from which the emollient particles are made.

To produce the tanning effect to skin, the visual effects material carried by the emollient particles of the invention constitutes self-tanning material. A preferred self-tanning material is dihydroxyacetone. Other self-tanning material or combinations of self-tanning materials can be used without departing from the invention. Emollient particles incorporating self-tanning material, in accordance with the principle of the invention, preferably incorporate approximately ten percent (10%) by weight of the self-tanning material, and less or more can be used depending on specific needs. Emollient particles incorporating self-tanning material are capable of being rubbed onto or otherwise applied to the skin, in which they will dissipate onto the skin leaving disposed thereon a persistent emollient layer and the self-tanning material, providing a tanning effect to the skin.

Preferably, emollient particles incorporating self-tanning material in accordance with the invention are disposed in a topical sunscreen preparation that is one of transparent and translucent, such as any variety of well-known aqueous or oil-based topical sunscreen preparations or the like. Disposing emollient particles incorporating self-tanning material as herein provided into such a topical sunscreen preparation provides the topical sunscreen preparation with self-tanning functionality without causing the undesirable effect of eliminating the clarity, transparency or translucency of the topical sunscreen preparation. Rather, the emollient spheres appear as particulate matter suspended in the topical sunscreen preparation or can be made to suspend with minor adjustments of the topical sunscreen preparation formulation by one skilled in the art. Because most transparent and translucent topical sunscreen preparations incorporate suspension agents, emollient particles incorporating self-tanning material in accordance with the invention will suspend in such topical sunscreen preparations. Additional suspension agents can be added, if desired.

Preferably, a sunscreen composition consisting of a mixture of a topical sunscreen preparation, which is one of transparent and translucent, and emollient particles carrying self-tanning material as provided herein incorporate ninety percent (90%) by weight of the topical sunscreen preparation and ten percent (10%) by weight of the emollient particles that incorporate the self-tanning material. Other percentages can be used depending on specific needs and depending on the desired self-tanning effect. A topical sunscreen preparation, which is one of transparent and translucent, having disposed therein emollient particles incorporating self-tanning material is capable of being rubbed onto or otherwise applied to the skin, in which the topical sunscreen preparation will absorb into the skin and the emollient particles will dissipate onto the skin, leaving disposed thereon the sunscreen material of the translucent topical sunscreen preparation, a persistent emollient layer and the self-tanning material of the emollient particles, protecting the skin from the harmful effects of prolonged exposure to UV radiation and imparting a desirable tanning effect to the skin. As previously intimated, emollient particles incorporating self-tanning material can also incorporate active sunscreen material as well, each at a desired percentage by weight of the preparation from which the emollient particles are formed, and used either independently or incorporated into a translucent topical sunscreen preparation.

To produce the coloring effect to skin, the visual effects material carried by the emollient particles of the invention constitutes cosmetic skin-coloring pigment or coloring material. Preferred coloring material include bronze coloring or pigment, copper coloring or pigment, iron oxides, and FD&C lakes, etc. Other colors can be used whether blue, red, orange, etc., and other suitable and well-known skin-coloring materials or pigments or skin-coloring agents such as paprika can be used without departing from the invention. Emollient particles incorporating coloring material, in accordance with the principle of the invention, preferably incorporate approximately ten percent (10%) by weight of the coloring material, and less or more can be used depending on specific needs. Emollient particles incorporating coloring material are capable of being rubbed onto or otherwise applied to the skin, in which they will dissipate onto the skin leaving disposed thereon a persistent emollient layer and the coloring material, providing a coloring effect to the skin.

Preferably, emollient particles incorporating coloring material in accordance with the invention are disposed in a topical sunscreen preparation, which is one of transparent and translucent, such as any variety of well-known aqueous or oil-based transparent or translucent topical sunscreen preparations or the like. Disposing emollient particles incorporating coloring material as herein provided into such a topical sunscreen preparation provides the topical sunscreen preparation with skin-coloring functionality without causing the undesirable effect of eliminating the clarity, transparency or translucency of the topical sunscreen preparation. Rather, the emollient spheres appear as particulate matter suspended in the topical sunscreen preparation or can be made to suspend with minor adjustments of the topical sunscreen preparation formulation by one skilled in the art. Because most transparent and translucent topical sunscreen preparations incorporate suspension agents, emollient particles incorporating coloring material in accordance with the invention will suspend in such topical sunscreen preparations. Additional suspension agents can be added, if desired.

Preferably, a sunscreen composition consisting of a mixture of a topical sunscreen preparation, which is one of transparent and translucent, and emollient particles carrying coloring material as provided herein incorporate ninety percent (90%) by weight of the translucent topical sunscreen preparation and ten percent (10%) by weight of the emollient particles that incorporate the coloring material. Other percentages can be used depending on specific needs and depending on the desired coloring effect. A topical sunscreen preparation, which is one of transparent and translucent, having disposed therein emollient particles incorporating coloring material is capable of being rubbed onto or otherwise applied to the skin, in which the topical sunscreen preparation will absorb into the skin and the emollient particles will dissipate onto the skin, leaving disposed thereon the sunscreen material of the topical sunscreen preparation, a persistent emollient layer and the coloring material of the emollient particles, protecting the skin from the harmful effects of prolonged exposure to UV radiation and imparting a desirable coloring effect to the skin. As previously intimated, emollient particles incorporating coloring material can also incorporate active sunscreen material as well, each at a desired percentage by weight of the preparation from which the emollient particles are formed, and used either independently or incorporated into a translucent topical sunscreen preparation.

To produce the glittery effect to skin, the visual effects material carried by the emollient particles of the invention constitutes glitter material. Preferred glitter material include any variety of readily available glitter material of one or more various colors. Emollient particles incorporating glitter material, in accordance with the principle of the invention, preferably incorporate approximately ten percent (10%) by weight of the glitter material, and less or more can be used depending on specific needs. Emollient particles incorporating glitter material are capable of being rubbed onto or otherwise applied to the skin, in which they will dissipate onto the skin leaving disposed thereon a persistent emollient layer and the glitter material, providing a glittery effect to the skin.

Preferably, emollient particles incorporating glitter material in accordance with the invention are disposed in a topical sunscreen preparation that is one of transparent and translucent, such as any variety of well-known aqueous or oil-based topical sunscreen preparations or the like. Disposing emollient particles incorporating glitter material as herein provided into such a topical sunscreen preparation provides the topical sunscreen preparation with skin-glitter functionality without causing the undesirable effect of eliminating the clarity, transparency or translucency of topical sunscreen preparation. Rather, the emollient spheres appear as particulate matter suspended in the topical sunscreen preparation or can be made to suspend with minor adjustments of the topical sunscreen preparation formulation by one skilled in the art. Because most transparent and translucent topical sunscreen preparations incorporate suspension agents, emollient particles incorporating glitter material in accordance with the invention will suspend therein. Additional suspension agents can be added, if desired.

Preferably, a sunscreen composition consisting of a mixture of a topical sunscreen preparation, which is one of transparent and translucent, and emollient particles carrying glitter material as provided herein incorporate ninety percent (90%) by weight of the translucent topical sunscreen preparation and ten percent (10%) by weight of the emollient particles that incorporate the glitter material. Other percentages can be used depending on specific needs and depending on the desired glitter effect. A topical sunscreen preparation, which is one of transparent and translucent, having disposed therein emollient particles incorporating glitter material is capable of being rubbed onto or otherwise applied to the skin, in which the topical sunscreen preparation will absorb into the skin and the emollient particles will dissipate onto the skin, leaving disposed thereon the sunscreen material of the topical sunscreen preparation, a persistent emollient layer and the glitter material of the emollient particles, protecting the skin from the harmful effects of prolonged exposure to UV radiation and imparting a desirable glittery effect to the skin. As previously intimated, emollient particles incorporating glitter material can also incorporate active sunscreen material as well, each at a desired percentage by weight of the preparation from which the emollient particles are formed, and used either independently or incorporated into a topical sunscreen preparation that is one of transparent and translucent.

The present invention is described above with reference to preferred embodiments. However, those skilled in the art will recognize that changes and modifications may be made in the described embodiments without departing from the nature and scope of the present invention. Various changes and modifications to the embodiments herein chosen for purposes of illustration will readily occur to those skilled in the art. To the extent that such modifications and variations do not depart from the spirit of the invention, they are intended to be included within the scope thereof.

Having fully described the invention in such clear and concise terms as to enable those skilled in the art to understand and practice the same, the invention claimed is:

1. A composition comprising:
emollient particles; and
active sunscreen material carried by the emollient particles.

2. The composition of claim 1, further comprising:
a topical sunscreen preparation that is one of transparent and translucent; and
the emollient particles disposed in the topical sunscreen preparation.

3. The composition of claim 1, the active sunscreen material comprising titanium dioxide.

4. The composition of claim 1, the active sunscreen material comprising zinc oxide.

5. The composition of claim 1, further comprising visual effects material carried by the emollient particles capable of imparting a visual characteristic to skin.

6. The composition of claim 5, wherein the visual characteristic comprises a tanning effect.

7. The composition of claim 5, wherein the visual characteristic comprises a coloring effect.

8. The composition of claim 5, wherein the visual characteristic comprises a glittery effect.

9. The composition of claim 1, further comprising self-tanning material carried by the emollient particles.

10. The composition of claim 9, the self-tanning material comprising dihydroxyacetone.

11. The composition of claim 1, further comprising coloring material carried by the emollient particles.

12. The composition of claim 1, further comprising glitter material carried by the emollient particles.

13. A composition comprising:
a topical sunscreen preparation that is one of transparent and translucent;
emollient particles disposed in the topical sunscreen preparation; and
active sunscreen material carried by the emollient particles.

14. The composition of claim 13, the active sunscreen material comprising titanium dioxide.

15. The composition of claim 13, the active sunscreen material comprising zinc oxide.

16. The composition of claim 13, further comprising self-tanning material carried by the emollient particles.

17. The composition of claim 16, the self-tanning material comprising dihydroxyacetone.

18. The composition of claim 13, further comprising visual effects material carried by the emollient particles.

19. The composition of claim 18, the visual effects material comprising pigment material.

20. The composition of claim 18, the visual effects material comprising glitter material.

21. A composition comprising:
a topical sunscreen preparation that is one of transparent and translucent; and
emollient particles disposed in the topical sunscreen preparation and carrying at least one of a)active sunscreen material, b)self-tanning material, and c)visual effects material.

22. The composition of claim 21, the active sunscreen material comprising titanium dioxide.

23. The composition of claim 21, the active sunscreen material comprising zinc oxide.

24. The composition of claim 21, the self-tanning material comprising dihydroxyacetone.

25. The composition of claim 21, the visual effects material comprising pigment material.

26. The composition of claim 21, the visual effects material comprising glitter material.

27. A method comprising steps of:
providing a topical sunscreen preparation that is one of transparent and translucent;
providing emollient particles carrying active sunscreen material; and
disposing the emollient particles in the topical sunscreen preparation.

28. The method of claim 27, the active sunscreen material comprising titanium dioxide.

29. The method of claim 27, the active sunscreen material comprising zinc oxide.

30. The method of claim 27, further comprising the emollient particles further carrying self-tanning material.

31. The method of claim 30, the self-tanning material comprising dihydroxyacetone.

32. The method of claim 27, further comprising the emollient particles further carrying visual effects material.

33. The method of claim 32, the visual effects material comprising pigment material.

34. The method of claim 32, the visual effects material comprising glitter material.

35. A method comprising steps of:
providing a topical sunscreen preparation that is one of transparent and translucent;
providing emollient particles carrying at least one of a)active sunscreen material, b)self-tanning material, and c)visual effects material; and
disposing the emollient particles in the topical sunscreen preparation.

36. The method of claim 35, the active sunscreen material comprising titanium dioxide.

37. The method of claim 35, the active sunscreen material comprising zinc oxide.

38. The method of claim 35, the self-tanning material comprising dihydroxyacetone.

39. The method of claim 35, the visual effects material comprising pigment material.

40. The method of claim 35, the visual effects material comprising glitter material.
